(19) United States
(12) Patent Application Publication
(10) Pub. No.: US 2026/0141551 A1
(43) Pub. Date: May 21, 2026

(54) METHOD FOR ESTIMATING PUPIL SIZE

(71) Applicant: KONTIGO CARE AB, Uppsala (SE)

(72) Inventors: Andreas ZETTERSTRÖM, MÅNKARBO (SE); Gunnar DAHLBERG, UPPSALA (SE); Karl ANDERSSON, Vänge (SE)

(21) Appl. No.: 19/122,406

(22) PCT Filed: Oct. 27, 2023
(86) PCT No.: PCT/SE2023/051070

§ 371 Date: Apr. 18, 2025

(30) Foreign Application Priority Data
Oct. 28, 2022 (SE) 2251254-5

(51) Int. Cl. G06T 7/62; A61B 3/11; A61B 3/14; G06T 5/40; G06T 5/60; G06T 5/92; G06V 10/82; G06V 40/18

(52) U.S. Cl. CPC G06T 7/62; A61B 3/112; A61B 3/14; G06T 5/40; G06T 5/60; G06T 5/92; G06V 10/82; G06V 40/193; G06T 2207/10024; G06T 2207/10152; G06T 2207/20072; G06T 2207/20081; G06T 2207/20084; G06T 2207/30041

(57) ABSTRACT

Methods and devices enabling the study of the pupil of an eye are presented. In particular, the methods and devices relate to estimating the pupil size and its reaction to changes in light. The invention is particularly suited for dark eyes, where the contrast between pupil and iris is low. At least one eye image is acquired (110). Each acquired eye image is processed (120). The processing comprises estimation of a pupil size in the acquired image. It is furthermore determined (130) that the pupil size estimation has been completed.

### TECHNICAL FIELD

The invention relates to pupillometry, i.e. methods and devices enabling the study of the pupil of an eye. In particular, the invention relates to measuring the pupil size.

### BACKGROUND

In the field of pupillometry, the pupil size is commonly measured. One application is to investigate the pupil light reflex, where the pupil size is estimated continuously during a few seconds, and where a light source is illuminating the eye during a part of that time.

The determination of pupil size based on a video film can be made in different ways. One possibility is to rely on contrast changes between iris color and pupil color, as discussed in U.S. Pat. No. 11,026,571B2 and denoted the “gray level jump”. If the “gray level jump”, i.e. the contrast, is small or non-existing, the methodology described in U.S. Pat. No. 11,026,571B2 is no longer applicable and fails to estimate the pupil size.

Another possibility is to rely on convolutional neural-network based methods. Such methods are often denoted artificial intelligence and require training prior to use. Such technology has been demonstrated functional for use in pupil segmentation as evident in “Open-source pupil segmentation and gaze estimation in neuroscience using deep learning” as published by Yiu and co-authors in Journal of Neuroscience Methods 324 (2019) 108307. This disclosure only shows eyes with bright irises compared to the dark pupil.

Similarly, the disclosure “RITnet: Real-time Semantic Segmentation of the Eye for Gaze Tracking” published by Chaudhary and co-authors in 2019 IEEE/CVF International Conference on Computer Vision Workshop (ICCVW) (DOI: 10.1109/ICCVW.2019.00568) discloses use of a neural-network based method for segmenting the image of a pupil, also using showing only eyes with bright irises compared to the dark pupil.

Yet another case is “Pupil Size Prediction Techniques Based on Convolution Neural Network” published by Whang and co-authors in Sensors (Basel). 2021 August; 21(15): 4965. (doi: 10.3390/s21154965) [https://www.ncbi.nlm.nih.gov/pmc/articles/PMC8347913/]. Also in this case, only eyes with bright irises compared to the dark pupil are shown.

There is therefore still a need for a pupil size measuring method being compatible also with dark eyes.

### SUMMARY OF THE INVENTION

An object of the technology presented here is to improve the ability of determining the pupil size, also for instance in cases where the contrast between iris and pupil is low. This would correspond to measuring the pupil size on individuals with very dark irises.

The above object is achieved by methods and devices according to the independent claims. Preferred embodiments are defined in dependent claims.

In a first aspect, a method for processing images of an eye comprises acquiring at least one eye image. Each acquired eye image is processed. The processing comprises estimation of a pupil size in the acquired image. It is furthermore determined that the pupil size estimation has been completed. The processing of each acquired eye image comprises enhancing contrast using Contrast-Limited Adaptive Histogram Equalization (CLAHE), applying a multilayer neural network, trained for distinguishing a pupil from an iris, and fitting an ellipse to pupil region perimeters. The determining of that the pupil size estimation has been completed comprises computing an average confidence for pixels residing withing the fitted ellipse around the pupil region perimeter, and comparing the computed average confidence to a predetermined value.

In a second aspect, a device for processing images of an eye comprises a camera for acquiring at least one eye image and a processor communicationally connected to the camera. The processor is configured for processing each acquired eye image. The processing comprises estimating a pupil size in the acquired image. The processor is further configured for determining that the pupil size estimation has been completed. The processing of each acquired eye image comprises enhancing contrast using Contrast-Limited Adaptive Histogram Equalization (CLAHE), applying a multilayer neural network, trained for distinguishing a pupil from an iris, and fitting an ellipse to pupil region perimeters. The determining of that the pupil size estimation has been completed comprises computing an average confidence for pixels residing withing the fitted ellipse around the pupil region perimeter, and comparing the computed average confidence to a predetermined value.

The invention is thus based on a novel data extraction method, optionally combined with a data confirmation procedure.

One advantage with the present technology is that even images of dark eyes can be processed.

### BRIEF DESCRIPTION OF THE DRAWINGS

FIG. 1 is a schematic flow diagram of steps of an embodiment of a method for processing images of an eye;

FIG. 2 is a schematic flow diagram of steps of another embodiment of a method for processing images of an eye;

FIG. 3 is a flow chart of steps of an embodiment of a single frame image processing algorithm of a method for processing images of an eye;

FIG. 4 is an illustration of grayscale values along a line across a high contrast eye and a low contrast eye, respectively;

FIG. 5 is an illustration of the pupil response to illumination; and

FIG. 6 is a schematic illustration of an embodiment of a device for processing images of an eye.

### DETAILED DESCRIPTION OF THE INVENTION

Embodiments of the present invention are further described below with reference to the accompanying drawings.

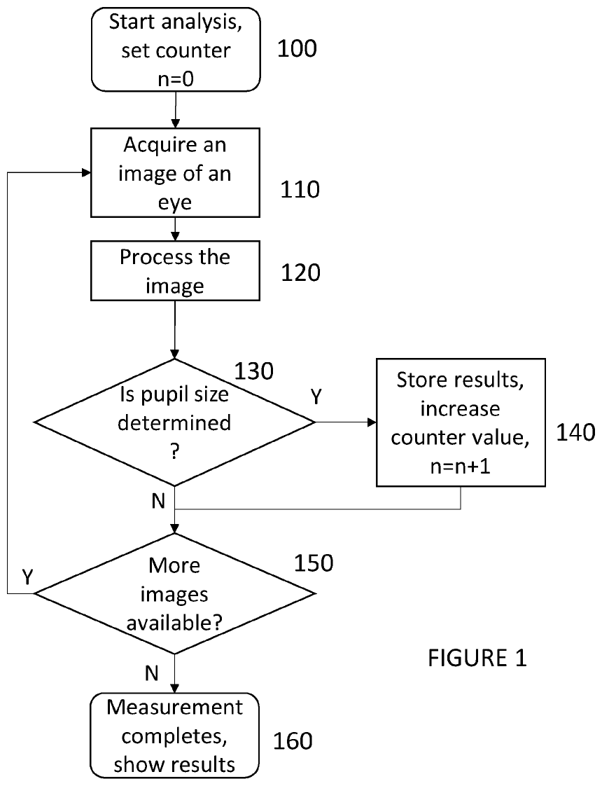

As shown in FIG. 1, the present technology relates to an image processing method based on machine vision, including the steps of: acquiring eye images 110, carrying out processing of each image respectively 120, and completing pupil size estimation 130.

In an embodiment, the process of the method is as follows:

To start with, in step 100, the system is initialized and the counter is cleared, i.e., set to be n=0. An image is made available to the method in step 110, for example by acquiring it by a camera, and the image is stored in a file format with high resolution, such as a bmp format.

In the steps 120 that follows, the image acquired by the camera is processed, and in the single-frame image processing process, several common situations may cause inaccurate pupil size determination. For example, the eye may be closed. Another example is that there are reflections in the location of the eye due to nearby light sources. Still another example is that the iris is dark in comparison to the pupil.

Therefore, the image is processed through the steps of (a) optionally converting the image to grayscale, (b) making images brighter using gamma correction, (c) enhancing contrast using Contrast-Limited Adaptive Histogram Equalization (CLAHE), (d) applying a multilayer neural network, trained for distinguishing the pupil from the iris, and (e) fitting an ellipse to the pupil/iris region perimeters. This will be further illustrated below.

As illustrated by step 130, if the image processing produced a pupil size determination, the result is stored and the counter is increased in step 140. If there are additional images to process, as concluded in step 150, the process is repeated from step 110, else the measurement is complete and results are shown in step 160.

In other words, in one embodiment, a method for processing images of an eye, comprises a step of acquiring at least one eye image. Each acquired eye image is processed. The processing comprises estimating a pupil size in the acquired image. The step of processing each acquired eye image comprises a number of part steps. The images are optionally converted to grayscale. Each acquired eye image is brightening using gamma correction. Contrast is enhanced using Contrast-Limited Adaptive Histogram Equalization (CLAHE). A multilayer neural network, trained for distinguishing a pupil from an iris, is applied. An ellipse is fitted to pupil region perimeters. Thereafter, it is determined that the pupil size estimation has been completed. This step in turn comprises computing of an average confidence for pixels residing withing the fitted ellipse around the pupil region perimeter. The computed average confidence is compared to a predetermined value.

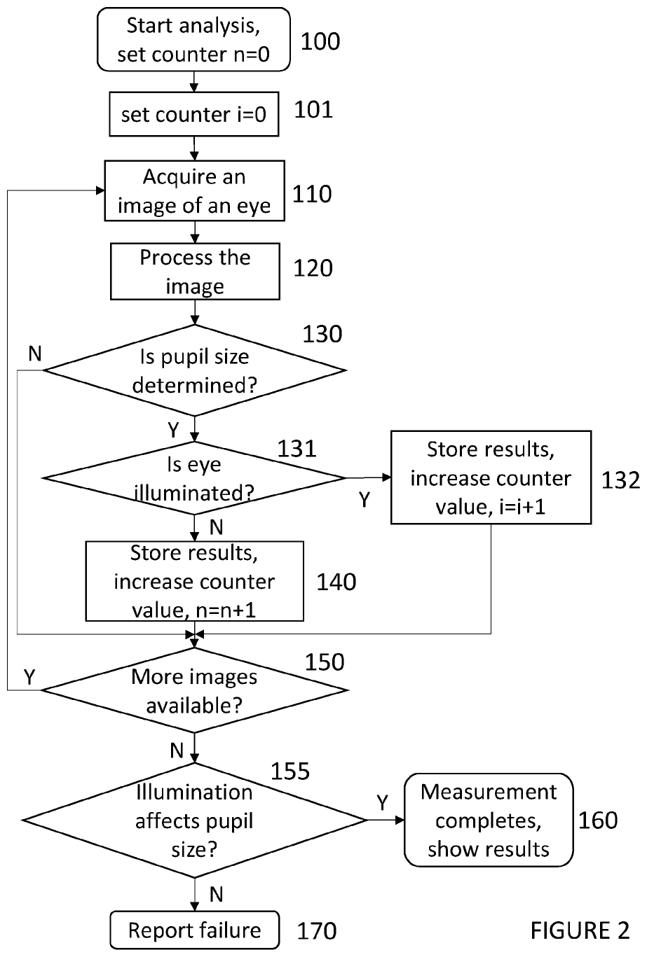

In another embodiment, as illustrated in FIG. 2, the process of the method is as follows: Here, a step of illuminating the eyes that are imaged using visible light during a part of the measurement sequence. To start with, the system is initialized and two counters are cleared, i.e., set to be n=0 in step 100; i=0 in step 101. An image made available to the method in step 110, in the same manner as in FIG. 1. In the steps that follow 120, the image acquired by the camera is processed in the same manner as in FIG. 1.

If the image processing produced a pupil size determination 130, the illumination status is determined in step 131. This can for example be done either by analyzing differences in luminance of consecutive images or it can be associated with hardware, where the camera and the illumination resides in one device, such as a mobile phone, and hence can tell if illumination was active or not. For illuminated images, results are stored and the illuminated counter is increased one unit in step 132, else results are stored as non-illuminated and the corresponding counter is increased one unit in step 140.

If there are additional images to process, as concluded in step 150, the process is repeated from step 110, else the process proceeds to estimate the effect of illumination on the pupil size, in step 155. If there is a noticeable reaction of estimated pupil size to illumination the measurement is complete and results are shown in step 160 else an error is reported in step 170.

In other words, in one embodiment, the step of acquiring at least one eye image comprises acquiring at least two eye images. A first image is captured at a first illumination level of the eye that is being imaged, and a, subsequent, second image is captured at a second illumination level of the eye that is being imaged. The second illumination level is higher than the first illumination level. A time between the acquisition of the first and second images is greater than 0.2 seconds and less than 5 seconds. The step of determining that the pupil size estimation has been completed further comprises comparing the estimated pupil size in the first and second image, and requiring that the pupil size changes more than a predefined value as a response to the change in illumination level.

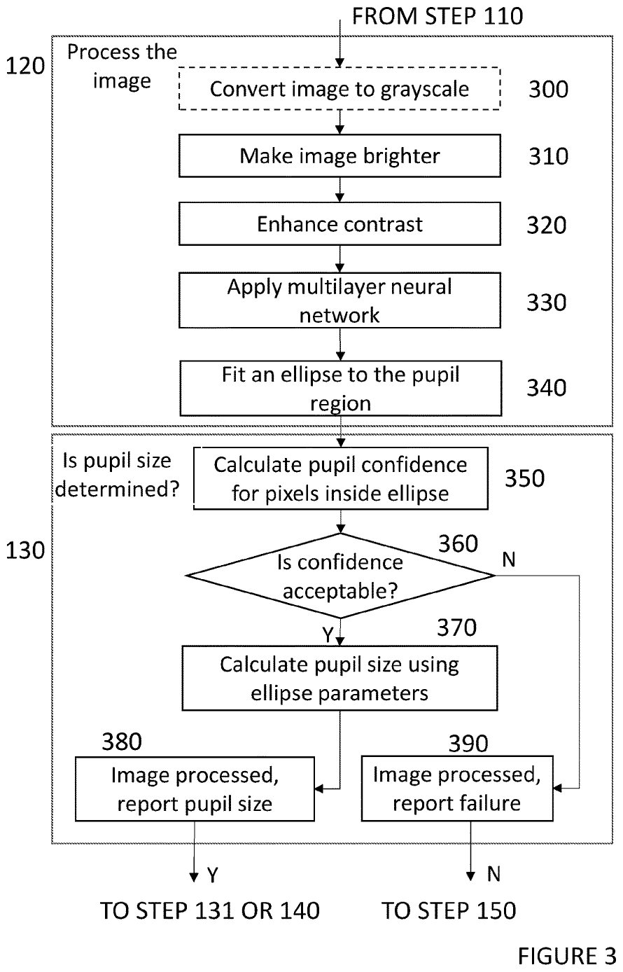

Referring now to FIG. 3, where an embodiment of the image processing step 120 and the determining step 130 are described in more detail.

To begin with, in step 300, the image is optionally converted to grayscale. One possible method is to convert the red-green-blue (RGB) code to grayscale using the formula Y=0.2989 R+0.5870 G+0.1140 B for each pixel. The image is then converted from three channels (RGB) to a single grayscale channel (Y).

Another possible method is to convert the red-green-blue (RGB) code to grayscale using a method which does not provide the most adequate grayscale representation from a human eye perspective, but rather provide a grayscale representation that distinguishes dark colors. In practice, this often means that the red channel should contribute more to the grayscale representation.

One such non-limiting example would be to use the formula Y=0.4392 R+0.4696 G+0.0912 B for each pixel.

Yet another possible method is to use CIELAB color coordinates. CIELAB is a well-known color space defined by the International Commission on Illumination in 1976. CIELAB coordinates have two representations for color and one for light intensity. As a non-limiting example it would be possible to use Y=CIELAB-A color coordinate for each pixel.

Still another possible method is to rely on a multilayer neural network that is adapted for color images. In that case the need for conversion to grayscale is not necessary, i.e. step 300 is omitted. In other words, a multilayer neural network is used, trained for, based on color images, distinguishing a pupil from an iris.

Next, in step 310, the image is made brighter using gamma correction. One possible method is to apply the formula Y′=255*(a/255)\hat{ }k where a is the grayscale channel input pixel value and Y′ is the brighter output. Gamma correction is hence a non-linear transformation of every pixel value in the image. By applying an exponent k lower than 1 the brightness of the image is increased. It is advisable that the exponent k is in the range of [0.6-0.95].

Thereafter, in step 320, the contrast of the image is enhanced using Contrast-Limited Adaptive Histogram Equalization (CLAHE), at least for grayscale images. CLAHE has been described in “Adaptive histogram equalization and its variations” by Pizer and co-authors as published in Computer Vision, Graphics, and Image Processing, Volume 39, Issue 3, 1987, Pages 355-368, https://doi.org/10.1016/S0734-189X(87)80186-X. CLAHE is operating on tiles, i.e. small subsets of the image, often denoted tiles. The tile size should be small, in the order of 10*10 pixels. CLAHE is necessary for grayscale images. For color images, CLAHE can either be omitted, or be conducted on each color channel (for example red, green and blue in RGB space).

Next, a multilayer neural network, trained for distinguishing the pupil from the iris, is applied in step 330. Procedures for establishing such a neural network has been discussed in the past, for example in “Pupil Size Prediction Techniques Based on Convolution Neural Network” published by Whang and co-authors in Sensors (Basel). 2021 August; 21(15): 4965. (doi: 10.3390/s21154965). The multilayer neural network is trained using grayscale images or color images, in dependence on whether step 300 is performed or not. It is preferable to employ a segmentation network which able to distinguish pupil and iris pixels from background pixels. This could encompass a network that consists of a U-net architecture with an encoder and decoder part, where the encoder is a Mobilenetv3 network pre-trained on imagenet images (https://www.image-net.org/). The network should be configured to produce three output probability values per pixel of the pixel being an iris, or a pupil or a background pixel. The network shall be configured to produce an output probability value per pixel of the pixel being a pupil.

Finally, in step 340, an ellipse is fitted to the pupil region perimeters. An ellipse is described as a parameter equation as follows:

$$x = z 1 + r 1 cos φ$$

$$y = z 2 + r 2 sin φ$$

wherein (z1, z2) is the coordinate of the ellipse center, (r1, r2) are the major and minor axes of the ellipse, and where φ is an angle. The cartesian coordinates (x,y) for the rim of the ellipse are obtained by processing a large number of angles φ from 0 to 360 degrees. In the ellipse fitting procedure, (z1,z2) and (r1,r2) are iteratively altered to produce the closest possible match of the ellipse representation as compared to the pupil region perimeter.

At this stage, there is a suggested region of the image that would constitute the pupil. In order to verify that so is the case, step 130 comprises that a first level of quality control is applied in step 350. This is conducted as calculating the average confidence (as provided by the neural network) for all pixels inside the suggested region. If the average confidence is greater than a predetermined value, as determined in step 360, the imaging process is considered quality assured at a first level and the size of the pupil is calculated using ellipse parameters in step 370, i.e. as derived from the fitted ellipse. The calculations are in step 380 used to deliver a pupil size result. Else an error is reported in step 390.

The process shown in FIG. 3 can be adapted to determine the iris size. The major difference would be that the neural network instead is trained for identifying iris (alternatively being trained to identify both iris and pupil at the same time), and the ellipse being fitted to the iris contour. From a general standpoint, determination of the iris size is less difficult because of its contrast to the whites of the eye (the sclera).

In one embodiment, the average confidence is calculated based on a predicted probability that a pixel is pupil.

In an embodiment, the step 110 of acquiring an image is conducted using a camera intended for visible light. This could be a camera found for example in a consumer grade smartphone. When capturing images using a camera for visible light, such as a typical camera in a consumer grade smartphone, the image of an eye may contain reflexes. Such reflexes often overlap with the pupil, which complicates the determination of pupil size. The reflexes may have different shapes. Circular shape reflexes are for example common when a point source lamp is located near the person at the time of taking the image. Rectangular shape reflexes are common when a computer screen is located near the person at the time of taking the image.

A difficult condition is when the eye is affected by corneal arcus (also known as arcus senilis, gerontoxon, arcus lipoides, arcus corneae, corneal arcus, arcus adiposus), which are rings in the peripheral cornea. It is usually caused by cholesterol deposits. In practice, the effect is that iris contains concentric different colored circles that may be mistaken for pupil by an automated algorithm that determines pupil size. Corneal arcus is particularly challenging in subjects with dark eyes, because it presents the combination of the low contrast between the dark iris and the dark pupil, and concentric circles.

Still another difficult condition is when the eye is affected by cloudy cornea, which is a loss of transparency of the cornea. Visually it appears as clouds in the otherwise transparent cornea. With such a condition, the edge between the pupil and the iris may become blurred which in turn may become a challenge for an automated algorithm that determines pupil size. Cloudy cornea is particularly challenging in subjects with dark eyes, because it may blur an already difficult-to-determine low contrast edge between the dark iris and the dark pupil.

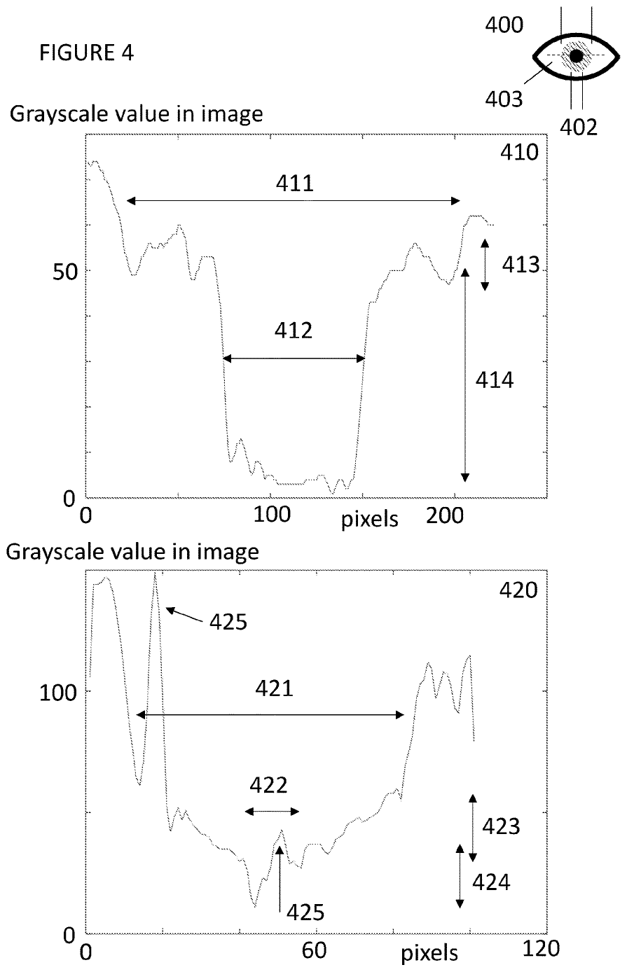

Referring now to FIG. 4, wherein the grayscale values of a cross-section of an image of eyes are shown. In each graph, an eye 400 with an iris width 401 and a pupil width 402 is depicted as grayscale values along a line crossing the center of the pupil 403, dashed line. Graph 410 corresponds to an eye found in FIG. 3 (top left image) in the publication “Pupil Size Prediction Techniques Based on Convolution Neural Network” published by Whang and co-authors in Sensors (Basel). 2021 August; 21(15): 4965. (doi: 10.3390/s21154965). This image depicts an eye with visible but light-colored iris. The iris width is indicated with arrow 411, and the pupil is indicated with arrow 412. The approximate grayscale variation of the iris is shown as arrow 413, and the approximate difference between the iris grayscale and the pupil grayscale is shown as arrow 414. We denote the entity “pupil-to-iris compared to iris color variation” PTI/ICV″. Arrow 414 is about 5 times longer than arrow 413, meaning pupil-to-iris compared to iris color variation is about 5; PTI/ICV≈5. This eye has well defined, distinct boundaries between pupil and iris, making it easier for any algorithm that aims at detecting pupil size.

Graph 420 shows a more difficult case. The iris 421 and the pupil 422 have essentially the same color resulting in about the same grayscale values. There are furthermore two reflections in this image; a light source that results in bright spots 425, two locations, that should be disregarded. The fluctuation of the iris grayscale 423 is about the same as the difference between average iris grayscale and average pupil grayscale 424 meaning PTI/ICV≈1. It is clear that PTI/ICV for the eye in graph 420 is less than 2. The present invention is capable of determining pupil size for both these eyes. Hence, the present invention can handle images of eyes where the variation of grayscale in the iris region is approximately the same as the average difference in grayscale of the iris compared to the pupil, meaning PTI/ICV<2 or PTI/ICV≈1. The grayscale values are deduced from a grayscale image or, as discussed below, a converted color image.

Even though the concept of PTI/ICV was developed for grayscale images, the principle can be applied to color images. The real-world problem lies in the estimation of pupil size in a situation where the color of iris and pupil are close to identical. This problem is equally challenging in a color image and a grayscale image. For the purpose of diagnosing the level of difficulty of a color image of an eye, the color image can be converted to grayscale for estimating PTI/ICV prior to submitting the color image to a multi-layer neural network.

In one embodiment, the iris and the pupil of the eye in the images have essentially the same color.

In one embodiment, the difference of average grayscale values of iris and pupil in the images is less than two times the variation of the grayscale values inside the iris region.

In one embodiment, the difference of average grayscale values of iris and pupil in the images is less than the variation of the grayscale values inside the iris region.

In one embodiment, a size of the iris is determined and the pupil size is expressed as a fraction of the iris size.

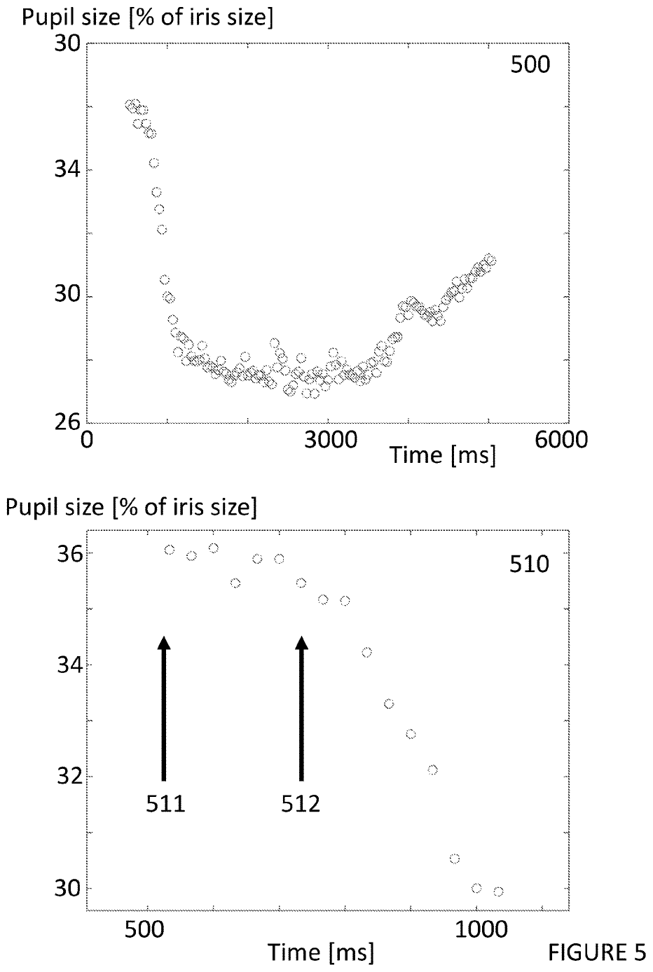

FIG. 5 shows a typical reaction pattern of a pupil which is illuminated. Graph 500 shows the pupil size (expressed as % iris size) over about 5 second time. Images were captured using the back camera of a smartphone. At time=530 ms the led-lamp was turned on and was kept on during 5 seconds. The led-lamp of the smartphone when located about 20-30 cm from the face resulted in about 300-400 lux of illumination. Shortly thereafter, the pupil contracts as a response to the elevated incident light. The contraction is completed after about 0.5 s in this particular case. The pupil often, but not always, contracts too much and hence adjust the size to a slightly larger size after another few seconds, which is the case in graph 500.

Graph 510 contains exactly the same data as graph 500, but is a magnification of the time until 1100 ms. Arrow 511 depicts the approximate timepoint when the led-lamp was turned on. Arrow 512 indicates the approximate timepoint when the pupil starts to contract. In this case, it takes about 200 ms for the pupil to react on the changed illumination condition. Hence, in a case where the pupil reaction to light is used as a quality assuring control, the time between a first image without illumination and a later image with illumination has to exceed 200 ms and should be smaller than 5 seconds.

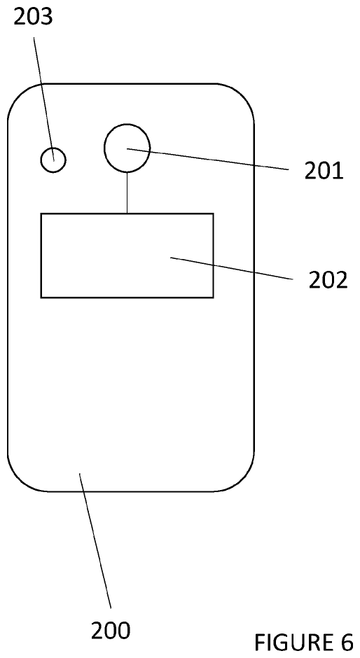

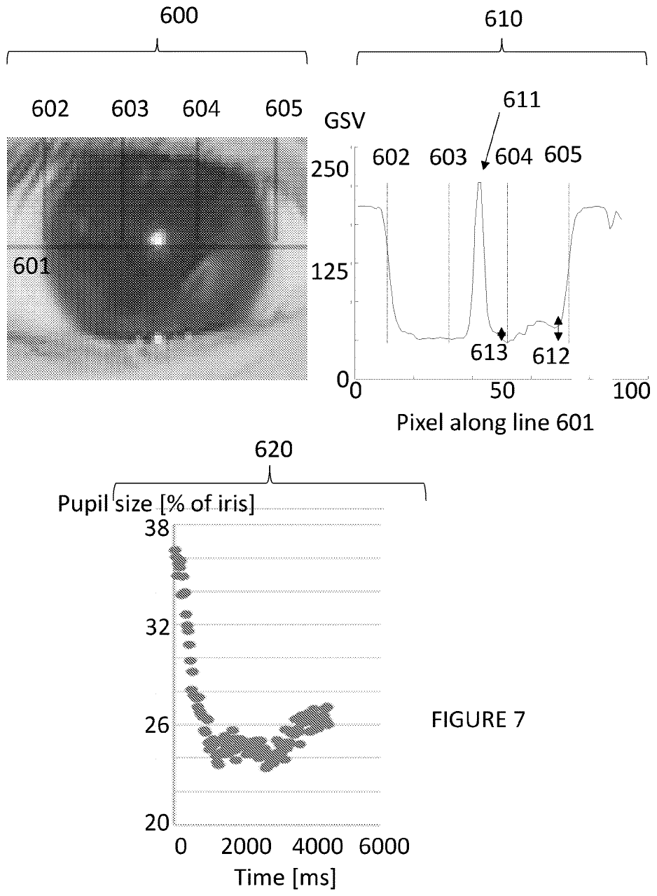

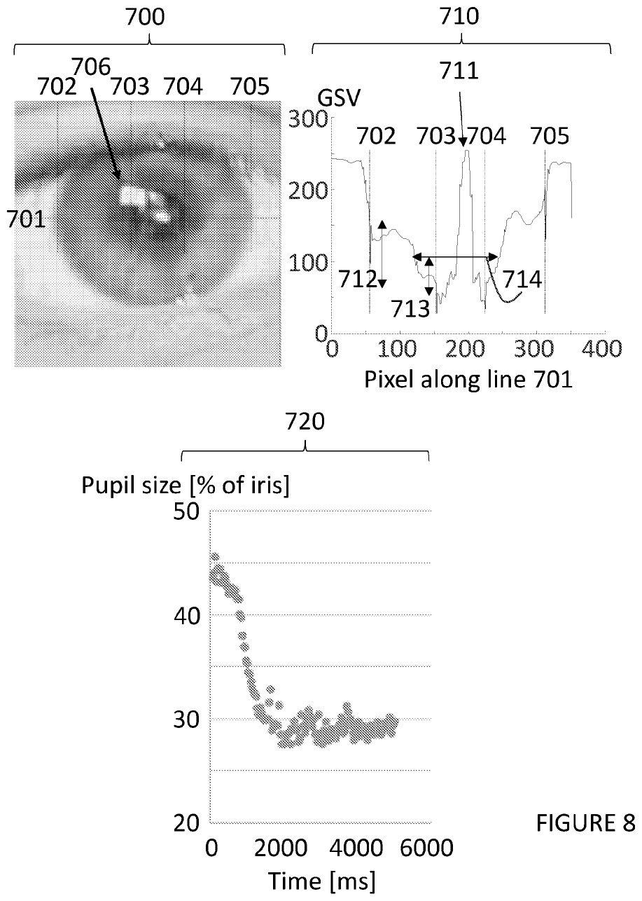

FIG. 6 is a schematic illustration of a device 200 for processing images of an eye. The device 200 comprises a camera 201 for acquiring at least one eye image. The device further comprises a processor 202 communicationally connected to the camera 201. The processor 202 is configured for processing each acquired eye image. The processing comprises estimating a pupil size in the acquired image. The processor is further configured for determining that the pupil size estimation has been completed. The processing of each acquired eye image comprises:
optionally converting the image to grayscale;
brightening each acquired eye image using gamma correction;
enhancing contrast using Contrast-Limited Adaptive Histogram Equalization—CLAHE;
applying a multilayer neural network, trained for distinguishing a pupil from an iris; and
fitting an ellipse to pupil region perimeters.

The determining of that the pupil size estimation has been completed comprises:
computing an average confidence for pixels residing withing the fitted ellipse around the pupil region perimeter; and
comparing the computed average confidence to a predetermined value.

In one embodiment, the device is a mobile phone.

In one embodiment, the device 200 further comprising a led-lamp 203.

### Example 1

Referring now to FIG. 7, where a grayscale image 600 of an eye with very dark pupil is described. In this example, the method of this invention was applied without using the optional step 300, i.e. color images were provided to a multilayer neural network. The method of this invention can correctly estimate the size of the pupil in this image. Pixel by pixel grayscale values GSV along horizontal line 601 are depicted in graph 610. The outer iris limits are indicated with vertical lines 602, 605 and the pupil limits are indicated with vertical lines 603, 604. There is a circular reflex 611 overlapping with the pupil, seen as a peak in the graph 610 and a white spot in the image 600. The variation of grayscale value in the iris is indicated with arrow 612. The difference between the average grayscale value of the iris and the average grayscale value of the pupil, excluding the reflex, is shown as arrow 613. Arrow 613 is less than half of arrow 612, meaning pupil-to-iris compared to iris color variation is less than 1; PTI/ICV<1. It is estimated that PTI/ICV is approximately 1/3. A pupillogram, i.e. pupil size over time, captured for a pupillary light reflex 620 shows that the pupil size responds as expected to illumination of the eye. This measurement was conducted using a consumer grade smartphone (an Iphone 13 mini) where the individual was videofilming the eyes using the back camera, and where the flashlight of the camera was turned on to illuminate the eyes after approximately 500 ms after the start of video capture. Upon being illuminated, the pupils are expected to contract, which is seen as a steep reduction in pupil size during the first 1000-1500 milliseconds of the video. After that the pupil size stabilizes because the size is adequate for the new brighter light condition caused by the flashlight.

Hence, the method of this invention can accurately determine pupil size in an image with PTI/ICV<1 and a visible reflex overlapping with the pupil.

### Example 2

Referring now to FIG. 8, where a grayscale image 700 of an eye with corneal arcus and cloudy cornea is described. In this example, the method of this invention was applied using the optional step 300, i.e. grayscale images were provided to a neural network. The method of this invention can correctly estimate the size of the pupil in this image. Pixel by pixel grayscale values GSV along horizontal line 701 are depicted in graph 710. The outer iris limits are indicated with vertical lines 702, 705 and the pupil limits are indicated with vertical lines 703, 704. There is a circular reflex 711 overlapping with the pupil, seen as a peak in the graph 710 and a white spot in the image 700. There is also a rectangular reflex 706 which overlaps with the edge of the pupil in a limited sector. Corneal arcus results in the iris having two concentrical grayscale levels, indicated by arrow 714. The variation of grayscale value in the iris is indicated with arrow 712 and is large because of corneal arcus. The difference between the average grayscale value of the iris and the average grayscale value of the pupil, excluding the circular reflex, is shown as arrow 713. Arrow 713 is shorter than arrow 712, meaning that pupil-to-iris compared to iris color variation is less than 1; PTI/ICV<1. It is estimated that PTI/ICV is approximately 1/2. In this case, the pupil is visible for a human, but any machine learning method would struggle with distinguishing the grayscale jump induced by corneal arcus and the grayscale jump caused by the iris-to-pupil edge. A pupillogram 720, i.e. pupil size over time, captured in the same manner as in example 1 shows that the pupil size responds as expected to illumination of the eye, which in turn indicates that the pipul size determination anyway is accurate.

Hence, the method of this invention can accurately determine pupil size in an image with PTI/ICV<1 where the eye has corneal arcus and two different types of reflexes overlapping with the pupil.

The embodiments described with reference to the drawings are exemplary and are intended to be illustrative of the invention and are not to be construed as limiting the invention. The scope of the invention is determined by the enclosed claims.

1. A method for processing images of an eye, comprising the steps of:
acquiring at least two eye images for the purpose of identifying a noticeable reaction of estimated pupil size to illumination;
wherein said eye presents Corneal arcus and/or Cloudy cornea;
wherein a first image is captured at a first illumination level of said eye that is being imaged, and a, subsequent, second image is captured at a second illumination level of said eye that is being imaged, wherein said second illumination level is higher than said first illumination level, and wherein a time between the acquisition of said first and second images is greater than 0.2 seconds and less than 5 seconds;
processing each acquired eye image, said processing comprises estimating a pupil size in the acquired image by applying a multilayer neural network, trained for distinguishing of a pupil from an iris; and
determining that said pupil size estimation has been completed,
wherein said step of determining that said pupil size estimation has been completed in turn comprises: computing an average confidence for pixels residing within said pupil region perimeter and comparing said computed average confidence to a predetermined value; and
wherein said step of determining that said pupil size estimation has been completed further comprises: comparing said estimated pupil size in said first and second image, and requiring that said pupil size changes more than a predefined value as a response to the change in illumination level.

2. The method as claimed in claim 1, wherein said step of processing each acquired eye image comprises:
brightening each acquired eye image using gamma correction;
if said images of an eye are grayscale images, enhancing contrast using Contrast-Limited Adaptive Histogram Equalization—CLAHE;
applying said multilayer neural network, trained for distinguishing of a pupil from an iris; and
fitting an ellipse to pupil region perimeters.

3. The method as claimed in claim 2, wherein said images of an eye are color images, wherein the method comprising the further step of:
converting said images to grayscale; and
enhancing contrast in said converted grayscale images using Contrast-Limited Adaptive Histogram Equalization—CLAHE;
wherein said neural network training is based on grayscale images.

4. The method as claimed in claim 1, wherein said iris and said pupil of said eye in said images have essentially the same color, wherein said same color of said iris and said pupil of said eye in said images is defined as, in said images, the difference of average grayscale values of iris and pupil being less than two times the variation of the grayscale values inside the iris region.

5. The method as claimed in claim 1, wherein in said images, the difference of average grayscale values of iris and pupil is less than two times the variation of the grayscale values inside the iris region, said grayscale values being deduced from a grayscale image or a converted color image.

6. The method as claimed in claim 1, wherein in said images, the difference of average grayscale values of iris and pupil is less than the variation of the grayscale values inside the iris region, said grayscale values being deduced from a grayscale image or a converted color image.

7. The method as claimed in claim 1, wherein a size of said iris is determined and where said pupil size is expressed as a fraction of said iris size.

8. The method as claimed in claim 1, wherein said average confidence is calculated based on a predicted probability that a pixel is pupil.

9. The method as claimed in claim 1, wherein said at least one eye image comprises reflexes overlapping with the pupil.

10. A device for processing images of an eye, comprising:
a camera for acquiring at least two eye images for the purpose of identifying a noticeable reaction of estimated pupil size to illumination; and
wherein said eye presents Corneal arcus and/or Cloudy cornea;
wherein a first image is captured at a first illumination level of said eye that is being imaged, and a, subsequent, second image is captured at a second illumination level of said eye that is being imaged, wherein said second illumination level is higher than said first illumination level, and wherein a time between the acquisition of said first and second images is greater than 0.2 seconds and less than 5 seconds;
a processor communicationally connected to said camera;
wherein said processor is configured for processing each acquired eye image, said processing comprises estimating a pupil size in the acquired image by applying a multilayer neural network, trained for distinguishing of a pupil from an iris; and
wherein said processor is further configured for determining that said pupil size estimation has been completed;
wherein said determining of that said pupil size estimation has been completed comprises computing an average confidence for pixels residing within the pupil region perimeter and comparing said computed average confidence to a predetermined value; and
wherein said determining of that said pupil size estimation has been completed further comprises comparing said estimated pupil size in said first and second image, and requiring that said pupil size changes more than a predefined value as a response to the change in illumination level.

11. The device as claimed in claim 10, wherein said processing of each acquired eye image comprises:
brightening each acquired eye image using gamma correction;
if said images of an eye are grayscale images, enhancing contrast using Contrast-Limited Adaptive Histogram Equalization—CLAHE;
applying a multilayer neural network, trained for distinguishing a pupil from an iris; and
fitting an ellipse to pupil region perimeters.

12. The device as claimed in claim 11, wherein said images of an eye are color images, wherein said processing of each acquired eye image further comprises converting said image to grayscale, enhancing contrast in said converted grayscale images using Contrast-Limited Adaptive Histogram Equalization—CLAHE, wherein said neural network training is based on grayscale images.

13. The device as claimed in claim 10, wherein said device is a mobile phone.

14. The device as claimed in claim 10, further comprising a led-lamp.

15. The method as claimed in claim 2, wherein said iris and said pupil of said eye in said images have essentially the same color, wherein said same color of said iris and said pupil of said eye in said images is defined as, in said images, the difference of average grayscale values of iris and pupil being less than two times the variation of the grayscale values inside the iris region.

16. The method as claimed in claim 3, wherein said iris and said pupil of said eye in said images have essentially the same color, wherein said same color of said iris and said pupil of said eye in said images is defined as, in said images, the difference of average grayscale values of iris and pupil being less than two times the variation of the grayscale values inside the iris region.

17. The method as claimed in claim 2, wherein in said images, the difference of average grayscale values of iris and pupil is less than two times the variation of the grayscale values inside the iris region, said grayscale values being deduced from a grayscale image or a converted color image.

18. The method as claimed in claim 3, wherein in said images, the difference of average grayscale values of iris and pupil is less than two times the variation of the grayscale values inside the iris region, said grayscale values being deduced from a grayscale image or a converted color image.

19. The method as claimed in claim 4, wherein in said images, the difference of average grayscale values of iris and pupil is less than two times the variation of the grayscale values inside the iris region, said grayscale values being deduced from a grayscale image or a converted color image.

20. The method as claimed in claim 15, wherein in said images, the difference of average grayscale values of iris and pupil is less than two times the variation of the grayscale values inside the iris region, said grayscale values being deduced from a grayscale image or a converted color image.
